(19) United States
(12) Patent Application Publication
(10) Pub. No.: US 2010/0221250 A1
(43) Pub. Date: Sep. 2, 2010

(54) METHODS OF TREATING BRAIN TUMORS WITH ANTIBODIES

(75) Inventors: Kyung Jin Kim, Cupertino, CA (US); John Laterra, Baltimore, MD (US); Bachchu Lal, Pikesville, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Galaxy Biotech, LLC, Cupertino, CA (US); Kennedy Krieger Institute, Inc., Baltimore, MD (US); Johns Hopkins University, Baltimore, MD (US)

(21) Appl. No.: 12/690,045

(22) Filed: Jan. 19, 2010

Related U.S. Application Data
(63) Continuation of application No. 11/446,045, filed on Jun. 1, 2006, now abandoned.
(60) Provisional application No. 60/687,118, filed on Jun. 2, 2005.
(60) Provisional application No. 60/751,092, filed on Dec. 15, 2005.

(51) Int. Cl. A61K 39/395; A61P 35/00

(52) U.S. Cl. 424/133.1; 424/141.1; 424/145.1

(57) ABSTRACT

The application is directed toward a method of treating a brain tumor in a patient comprising systemically administering a monoclonal antibody.

### CROSS-REFERENCE TO RELATED APPLICATIONS

The present application is a nonprovisional and claims the benefit of 60/687,118, filed Jun. 2, 2005 and 60/751,092 filed Dec. 15, 2005, both incorporated by reference in their entirety for all purposes.

### STATEMENT OF GOVERNMENT INTEREST

The work described in this application was funded in part by Grants 1R43CA101283-01A1 and RO1 NS32148 from the National Institutes of Health. The US government may have certain rights in this invention.

### FIELD OF THE INVENTION

The present invention relates generally to treatment of brain tumors with antibodies and more particularly, for example, to treatment of brain tumors with monoclonal antibodies that bind to and neutralize Hepatocyte Growth Factor.

### BACKGROUND OF THE INVENTION

Human Hepatocyte Growth Factor (HGF) is a multifunctional heterodimeric polypeptide produced by mesenchymal cells. HGF has been shown to stimulate angiogenesis, morphogenesis and motogenesis, as well as the growth and scattering of various cell types (Bussolino et al., J. Cell. Biol. 119: 629, 1992; Zarnegar and Michalopoulos, J. Cell. Biol. 129:1177, 1995; Matsumoto et al., Ciba. Found. Symp. 212:198, 1997; Birchmeier and Gherardi, Trends Cell. Biol. 8:404, 1998; Xin et al. Am. J. Pathol. 158:1111, 2001). The pleiotropic activities of HGF are mediated through its receptor, a transmembrane tyrosine kinase encoded by the proto-oncogene cMet. In addition to regulating a variety of normal cellular functions, HGF and its receptor c-Met have been shown to be involved in the initiation, invasion and metastasis of tumors (Jeffers et al., J. Mol. Med. 74:505, 1996; Comoglio and Trusolino, J. Clin. Invest. 109:857, 2002). HGF/cMet are coexpressed, often over-expressed, on various human solid tumors including tumors derived from lung, colon, rectum, stomach, kidney, ovary, skin, multiple myeloma and thyroid tissue (Prat et al., Int. J. Cancer 49:323, 1991; Chan et al., Oncogene 2:593, 1988; Weidner et al., Am. J. Respir. Cell. Mol. Biol. 8:229, 1993; Derksen et al., Blood 99:1405, 2002). HGF acts as an autocrine (Rong et al., Proc. Natl. Acad. Sci. USA 91:4731, 1994; Koochekpour et al., Cancer Res. 57:5391, 1997) and paracrine growth factor (Weidner et al., Am. J. Respir. Cell. Mol. Biol. 8:229, 1993) and anti-apoptotic regulator (Gao et al., J. Biol. Chem. 276:47257, 2001) for these tumors. Thus, antagonistic molecules, for example antibodies, blocking the HGF-cMet pathway potentially have wide anti-cancer therapeutic potential.

HGF is a 102 kDa protein with sequence and structural similarity to plasminogen and other enzymes of blood coagulation (Nakamura et al., Nature 342:440, 1989; Weidner et al., Am. J. Respir. Cell. Mol. Biol. 8:229, 1993, each of which is incorporated herein by reference). Human HGF is synthesized as a 728 amino acid precursor (preproHGF), which undergoes intracellular cleavage to an inactive, single chain form (proHGF) (Nakamura et al., Nature 342:440, 1989; Rosen et al., J. Cell. Biol. 127:1783, 1994). Upon extracellular secretion, proHGF is cleaved to yield the biologically active disulfide-linked heterodimeric molecule composed of an α-subunit and β-subunit (Nakamura et al., Nature 342:440, 1989; Naldini et al., EMBO J. 11:4825, 1992). The α-subunit contains 440 residues (69 kDa with glycosylation), consisting of the N-terminal hairpin domain and four kringle domains. The β-subunit contains 234 residues (34 kDa) and has a serine protease-like domain, which lacks proteolytic activity. Cleavage of HGF is required for receptor activation, but not for receptor binding (Hartmann et al., Proc. Natl. Acad. Sci. USA 89:11574, 1992; Lokker et al., J. Biol. Chem. 268:17145, 1992). HGF contains 4 putative N-glycosylation sites, 1 in the α-subunit and 3 in the β-subunit. HGF has 2 unique cell specific binding sites: a high affinity (Kd=2×10-10 M) binding site for the cMet receptor and a low affinity (Kd=10-9 M) binding site for heparin sulfate proteoglycans (HSPG), which are present on the cell surface and extracellular matrix (Naldini et al., Oncogene 6:501, 1991; Bardelli et al., J. Biotechnol. 37:109, 1994; Sakata et al., J. Biol. Chem., 272:9457, 1997). NK2 (a protein encompassing the N-terminus and first two kringle domains of the α-subunit) is sufficient for binding to cMet and activation of the signal cascade for motility, however the full length protein is required for the mitogenic response (Weidner et al., Am. J. Respir. Cell. Mol. Biol. 8:229, 1993). HSPG binds to HGF by interacting with the N terminus of HGF (Aoyama, et al., Biochem. 36:10286, 1997; Sakata, et al., J. Biol. Chem. 272:9457, 1997). Postulated roles for the HSPG-HGF interaction include the enhancement of HGF bioavailability, biological activity and oligomerization (Bardelli, et al., J. Biotechnol. 37:109, 1994; Zioncheck et al., J. Biol. Chem. 270:16871, 1995).

cMet is a member of the class IV protein tyrosine kinase receptor family. The full length cMet gene was cloned and identified as the cMet proto-oncogene (Cooper et al., Nature 311:29, 1984; Park et al., Proc. Natl. Acad. Sci. USA 84:6379, 1987). The cMet receptor is initially synthesized as a single chain, partially glycosylated precursor, p170(MET) (FIG. 1) (Park et al., Proc. Natl. Acad. Sci. USA 84:6379, 1987; Giordano et al., Nature 339:155, 1989; Giordano et al., Oncogene 4:1383, 1989; Bardelli et al., J. Biotechnol. 37:109, 1994). Upon further glycosylation, the protein is proteolytically cleaved into a heterodimeric 190 kDa mature protein (1385 amino acids), consisting of the 50 kDa α-subunit (residues 1-307) and the 145 kDa β-subunit. The cytoplasmic tyrosine kinase domain of the β-subunit is involved in signal transduction.

Several different approaches have been investigated to attempt to obtain an effective antagonistic molecule of HGF/cMET: truncated HGF proteins such as NK1 (N terminal domain plus kringle domain 1; Lokker et al., J. Biol. Chem. 268:17145, 1993), NK2 (N terminal domain plus kringle domains 1 and 2; Chan et al., Science 254:1382, 1991) and NK4 (N-terminal domain plus four kringle domains; Kuba et al., Cancer Res. 60:6737, 2000) and anti-cMet mAbs (Dodge, Master's Thesis, San Francisco State University, 1998).

Most recently, Cao et al. (Proc. Natl. Acad. Sci. USA. 98: 7443, 2001, which is incorporated herein by reference) reported that administration of a combination of 3 mAbs to HGF inhibited growth of subcutaneous glioma xenografts in mice. WO 2005/017107 A2, which is herein incorporated by reference in its entirety for all purposes, reported that treatment with a single anti-HGF mAb could inhibit growth of subcutaneous glioma xenografts in mice. However, these publications did not address the question of whether systemic administration of an anti-HGF or other mAb can inhibit growth of a tumor in the brain, where the blood-brain barrier presents obstacles (Rich et al., Nat. Rev. Drug Discov. 3: 430, 2004). Indeed, previously observed inefficacy of systemic antibody therapies against central nervous system (CNS) tumors has been attributed to restricted vascular permeability even for CNS metastases (Bendell et al., Cancer 97: 2972, 2003).

Thus, there is a need for a method to treat brain tumors by systemic administration of a mAb. The present invention fulfills this and other needs.

### BRIEF SUMMARY OF THE INVENTION

In one embodiment, the invention provides a method of treating a brain tumor in a patient by systemic administration of a mAb. The brain tumor may be a glioma such as an astrocytoma, e.g., a glioblastoma. Administration may be, for example, by intravenous, intramuscular or subcutaneous routes. In a preferred embodiment, the mAb is a neutralizing mAb to Hepatocyte Growth Factor (HGF) such as a humanized L2G7 mAb. In another preferred embodiment, systemic administration of a mAb such as a neutralizing anti-HGF mAb is used to induce regression of a brain tumor.

### BRIEF DESCRIPTION OF THE DRAWINGS

FIG. 1. Binding and blocking activities of anti-HGF mAbs measured by ELISA. A. For binding, mAbs were captured onto a goat anti-mouse IgG coated ELISA plate, blocked with BSA and incubated with HGF-Flag (1 μg/ml), followed by HRP-M2 anti-Flag mAb (Invitrogen). B. For blocking of HGF-Flag to Met-Fc binding, plates were coated with goat anti-human IgG-Fc, blocked with BSA, incubated with Met-Fc (2 μg/ml), and then with HGF-Flag (1 μg/ml) +/−anti-HGF mAbs. The bound HGF-Flag bound was detected with HRP-M2 anti-Flag mAb.

FIG. 2. Blocking effects of mAb L2G7 on the scattering, mitogenic, angiogenic, and anti-apoptotic activities of HGF. A. MDCK cells (ATCC) were stimulated with 50 ng/ml of HGF+/−10 μg/ml L2G7 for 2 days as described (Cao et al., Proc. Natl. Acad. Sci. USA. 98: 7443, 2001). Photographs were taken at 100× magnification after the cells were stained with crystal violet. B. My 1 Lu mink lung epithelial cells (ATCC; 5×104 cells/ml) were incubated in serum free DMEM with or without HGF (50 ng/ml) and L2G7 or isotype-matched control mAb (mIgG) for 24 hr, and the level of cell proliferation determined by addition of 3H-thymidine for 6 hr. C. As described (Xin et al. Am. J. Pathol. 158, 1111, 2001), HUVEC (CAMBREX; 104 cells/100 μl/well) were incubated in EBM-2/0.1% FCS with or without HGF (50 ng/ml) and L2G7 or control mAb for 72 hr and the level of proliferation determined by the addition of WST-1. D. As described (Xin et al. Am. J. Pathol. 158, 1111, 2001), HUVEC (6×104 cells/100 μl/well) in DMEM/gel were overlayed with 100 μl/well of EMB-2/0.1% FCS/0.1% BSA with or without 200 ng/ml of HGF+/−20 μg/ml of L2G7. After 48 hr incubation, cells were fixed and stained using toluidine blue and photographs taken at 40× magnification. E. As described (Fan et al. Oncogene 24: 1749, 2005), U87 tumor cells in serum free DMEM were treated with or without HGF (20 ng/ml)+/−mAb L2G7 (20 μg/ml) or isotype control antibody (mIgG) for 48 hr and then with anti-Fas mAb CH-11 (Upstate Biotechnology, 40 ng/ml) for 24 hr, and cell viability determined by the addition of WST-1. In b, c and e, values are mean+/−s.d.

FIG. 3. Inhibition or regression of glioma tumor xenografts by L2G7. U118 (A) or U87 (B) glioma tumor cells were implanted subcutaneously into NIH III Beige/Nude mice and tumor size monitored as described (Kim et al., Nature 362: 841, 1993). After tumor size had reached ˜50 mm3, groups of mice (n=6 or 7) were treated twice weekly i.p. with 50 or 100 μg L2G7 or 100 μg isotype-matched control mAb (mIgG) or PBS as indicated; arrows show first day of treatment. Values are mean tumor volume+/−s.e.m. C. U87 tumor cells (105 per mouse) were injected intracranially into the caudate/putamen of Scid/beige mice as described (Abounader et al. FASEB J. 16, 108, 2002). Starting and ending respectively on day 5 and day 52 as indicated by arrows, groups of mice (n=10) were administered i.p. 100 μg L2G7 or PBS twice weekly and survival monitored. Survival studies were analyzed by Kaplan-Meier plots. D. Brain sections prepared as described (Abounader et al. FASEB J. 16, 108, 2002) from representative mice sacrificed on day 21 after 3 doses of twice weekly i.p. treatment with 100 μg L2G7 or PBS, showing size of U87 intracranial xenografts. E. Intracranial U87 tumor volumes in individual mice on Day 18 before starting treatment and on Day 29 after treatment 3 times with L2G7. F. Brain sections from representative mice on Day 18 before treatment and on Day 29 after treatment with L2G7 or control mAb.

FIG. 4. Histological analysis of brain sections from mice with U87 intracranial xenografts. The mice were sacrificed after treatment of pre-established tumors with three twice-weekly doses of L2G7 or control. Perfusion-fixed cryostat sections were stained with H&E and the indicated antibody and indexes quantified using computer-assisted image analysis. A. Anti-Ki67 (DAKO) to detect proliferating cells. B. Anti-laminin (Life Technologies) to detect blood vessels. C. Antibody to cleaved caspase-3 (Cell Signaling Technology) to detect apoptotic tumor cell responses.

### DETAILED DESCRIPTION OF THE INVENTION

The invention provides a method of treating brain tumors by systemic administration of a neutralizing mAb to HGF or antibodies against other cytokines such as growth factors or against cell surface proteins such as cytokine receptors. Although an understanding of mechanism is not required for practice of the invention, it is believed that the success of the invention resides at least in part due to passage of antibody from the blood into brain tumors due to a defective blood brain barrier within the tumors.

### 1. Antibodies

Antibodies are very large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions fold up together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-D space to form the actual antibody binding site which locks onto the target antigen. The position and length of the CDRs have been precisely defined. Kabat, E. et al., Sequences of Proteins of Immunological Interest, U.S. Department of Health and Human Services, 1983, 1987. The part of a variable region not contained in the CDRs is called the framework, which forms the environment for the CDRs.

A monoclonal antibody (mAb) is a single molecular species of antibody and therefore does not encompass polyclonal antibodies produced by injecting an animal (such as a rodent, rabbit or goat) with an antigen, and extracting serum from the animal. A humanized antibody is a genetically engineered (monoclonal) antibody in which the CDRs from a mouse antibody (“donor antibody”, which can also be rat, hamster or other similar species) are grafted onto a human antibody (“acceptor antibody”). Humanized antibodies can also be made with less than the complete CDRs from a mouse antibody (e.g., Pascalis et al., J. Immunol. 169:3076, 2002). Thus, a humanized antibody is an antibody having CDRs from a donor antibody and variable region framework and constant regions from a human antibody. In addition, in order to retain high binding affinity, at least one of two additional structural elements can be employed. See, U.S. Pat. Nos. 5,530,101 and 5,585,089, each of which is incorporated herein by reference, which provide detailed instructions for construction of humanized antibodies.

In the first structural element, the framework of the heavy chain variable region of the humanized antibody is chosen to have maximal sequence identity (between 65% and 95%) with the framework of the heavy chain variable region of the donor antibody, by suitably selecting the acceptor antibody from among the many known human antibodies. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

As used herein, the term “human-like” antibody refers to a mAb in which a substantial portion of the amino acid sequence of one or both chains (e.g., about 50% or more) originates from human immunoglobulin genes. Hence, human-like antibodies encompass but are not limited to chimeric, humanized and human antibodies. As used herein, a “reduced-immunogenicity” antibody is one expected to have significantly less immunogenicity than a mouse antibody when administered to human patients. Such antibodies encompass chimeric, humanized and human antibodies as well as antibodies made by replacing specific amino acids in mouse antibodies that may contibute to B- or T-cell epitopes, for example exposed residues (Padlan, Mol. Immunol. 28:489, 1991). As used herein, a “genetically engineered” antibody is one for which the genes have been constructed or put in an unnatural environment (e.g., human genes in a mouse or on a bacteriophage) with the help of recombinant DNA techniques, and would therefore, e.g., not encompass a mouse mAb made with conventional hybridoma technology.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

### 2. Neutralizing Anti-HGF Antibodies

A monoclonal antibody (mAb) that binds HGF (i.e., an anti-HGF mAb) is said to neutralize HGF, or be neutralizing, if the binding partially or completely inhibits one or more biological activities of HGF (i.e., when the mAb is used as a single agent). Among the biological properties of HGF that a neutralizing antibody may inhibit are the ability of HGF to bind to its cMet receptor, to cause the scattering of certain cell lines such as Madin-Darby canine kidney (MDCK) cells; to stimulate proliferation of (i.e., be mitogenic for) certain cells including hepatocytes, 4 MBr-5 monkey epithelial cells, and various human tumor cells; or to stimulate angiogenesis, for example as measured by stimulation of human vascular endothelial cell (HUVEC) proliferation or tube formation or by induction of blood vessels when applied to the chick embryo chorioallantoic membrane (CAM). Antibodies used in the invention preferably bind to human HGF, i.e., to the protein encoded by the GenBank sequence with Accession number D90334. Similarly, a neutralizing, i.e., antagonist antibody against any cytokine or cytokine receptor may inhibit binding of the cytokine to the receptor and/or inhibit transmission of a signal to the cell by the cytokine. If the cytokine is a growth factor, such an antibody may inhibit proliferation of cells induced by the cytokine.

A neutralizing mAb used in the invention typically inhibits at a concentration of, e.g., 0.01, 0.1, 0.5, 1, 2, 5, 10, 20 or 50 μg/ml a biological function of a cytokine, e.g., HGF (for example, stimulation of proliferation or angiogenesis) by about at least 50% but preferably 75%, more preferably by 90% or 95% or even 99%, and most preferably approximately 100% (essentially completely) as assayed by methods described under Examples or known in the art. Typically, the extent of inhibition is measured when the amount of cytokine used is just sufficient to fully stimulate the biological activity, or is 0.05, 0.1, 0.5, 1, 3 or 10 μg/ml. Preferably, at least 50%, 75%, 90%, or 95% or essentially complete inhibition is achieved when the molar ratio of antibody to cytokine is 0.5×, 1×, 2×, 3×, 5× or 10×. Preferably, the mAb is neutralizing, i.e., inhibit the biological activity, when used as a single agent, but in some methods, two mAbs are used together to give inhibition. Most preferably, the mAb neutralizes not just one but several of the biological activities listed above; for purposes herein, an anti-HGF mAb that used as a single agent neutralizes all the biological activities of HGF is called “fully neutralizing”, and such mAbs are most preferable. MAbs used in the invention are preferably be specific for HGF, that is they do not bind, or only bind to a much lesser extent, proteins that are related to HGF such as fibroblast growth factor (FGF) and vascular endothelial growth factor (VEGF). The mAbs typically have a binding affinity (Ka) of at least 10⁷ M⁻¹ but preferably 10⁸ M⁻¹ or higher, and most preferably 10⁹ M⁻¹ or higher or even 10¹⁰ M⁻¹ or higher.

MAbs used in the invention include antibodies in their natural tetrameric form (2 light chains and 2 heavy chains) and may be of any of the known isotypes IgG, IgA, IgM, IgD and IgE and their subtypes, i.e., human IgG1, IgG2, IgG3, IgG4 and mouse IgG1, IgG2a, IgG2b, and IgG3. The mAbs are also meant to include fragments of antibodies such as Fv, Fab and F(ab′)₂; bifunctional hybrid antibodies (e.g., Lanzavecchia et al., Eur. J. Immunol. 17:105, 1987), single-chain antibodies (Huston et al., Proc. Natl. Acad. Sci. USA 85:5879, 1988; Bird et al., Science 242:423, 1988); and antibodies with altered constant regions (e.g., U.S. Pat. No. 5,624,821). The mAbs may be of animal (e.g., mouse, rat, hamster or chicken) origin, or they may be genetically engineered. Rodent mAbs are made by standard methods well-known in the art, comprising multiple immunization with HGF in appropriate adjuvant i.p., i.v., or into the footpad, followed by extraction of spleen or lymph node cells and fusion with a suitable immortalized cell line, and then selection for hybridomas that produce antibody binding to HGF, e.g., see under Examples. Chimeric and humanized mAbs, made by art-known methods mentioned supra, are used in preferred embodiments of the invention. Human antibodies made, e.g., by phage display or transgenic mice methods are also preferred (see e.g., Dower et al., McCafferty et al., Winter, Lonberg et al., Kucherlapati, supra). More generally, human-like, reduced immunogenicity and genetically engineered antibodies as defined herein are all preferred.

The neutralizing anti-HGF mAb L2G7 (deposited at the American Type Culture Collection under ATCC Number PTA-5162 according to the Budapest treaty) is a preferred example of a Mab for use in the invention. The deposit will be maintained at an authorized depository and replaced in the event of mutation, nonviability or destruction for a period of at least five years after the most recent request for release of a sample was received by the depository, for a period of at least thirty years after the date of the deposit, or during the enforceable life of the related patent, whichever period is longest. All restrictions on the availability to the public of these cell lines will be irrevocably removed upon the issuance of a patent from the application. Neutralizing mAbs with the same or overlapping epitope as L2G7 provide other examples. Variants of L2G7 such as a chimeric or humanized form of L2G7 are especially preferred. A mAb that competes with L2G7 for binding to HGF and neutralizes HGF in in vitro or in vivo assays described herein is also preferred. Other variants of L2G7 such as mAbs that are 90%, 95% or 99% identical to L2G7 in variable region amino acid sequence (e.g., when aligned by the Kabat numbering system; Kabat et al., op. cit.), at least in the CDRs, and maintain its functional properties, or which differ from it by a small number of functionally inconsequential amino acid substitutions (e.g., conservative substitutions), deletions, or insertions may also be used in the invention. Other preferred mAbs include human-like, reduced-immunogenicity and genetically engineered mAbs as defined herein.

Any amino acid substitutions from exemplified immunoglobulins are preferably conservative amino acid substitutions. For purposes of classifying amino acids substitutions as conservative or nonconservative, amino acids may be grouped as follows: Group I (hydrophobic sidechains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Yet other mAbs preferred for use in the invention include all the anti-HGF mAbs described in US 2005/0019327 A1 or WO 2005/017107 A2, whether explicitly by name or sequence or implicitly by description or relation to explicitly described mAbs (both cited applications are herein incorporated by reference for their disclosure of antibodies and all other purposes). Especially preferred mAbs are those produced by the hybridomas designated therein as 1.24.1, 1.29.1, 1.60.1, 1.61.3, 1.74.3, 1.75.1, 2.4.4, 2.12.1, 2.40.1 and 3.10.1 and respectively defined by their heavy and light chain variable region sequences provided by SEQ ID NO's 24-43 of WO2005/017107 A2; mAbs possessing the same respective CDRs as any of these listed mAbs; mAbs having light and heavy chain variable regions that are at least 90%, 95% or 99% identical to the respective variable regions of these listed mAbs or differing from them only by inconsequential amino acid substitutions, deletion or insertions; mAbs binding to the same epitope of HGF as any of these listed mAbs, and all mAbs encompassed by claims 1 through 94 therein. Sequence identities are determined between immunoglobulin variable region sequences aligned using the Kabat numbering convention.

In other embodiments, a mAb for use in the invention, i.e., for treatment of a brain tumor by systemic administration of the mAb, binds to one or more of the following growth factors: vascular endothelial cell growth factor (VEGF); a neurotrophin such as nerve growth factor (NGF), brain-derived neurotrophic factor (BDNF), or NT-3; a transforming growth factor such as TGF-alpha or TGF-beta (TGF-β1 and/or TGF-β2); platelet-derived growth factor (PDGF); epidermal growth factor (EGF); heregulin; epiregulin; emphiregulin; a neuregulin (NRG-1α and/or NRG-1β, NRG-2α and/or NRG-2β, NRG-3, or NRG-4), insulin-like growth factor (IGF-1 and IGF-2); or in a preferred embodiment a fibroblast growth factor (FGF) especially acidic FGF (FGF-1) or most preferably basic FGF (FGF-2), but alternatively FGF-n, where n is any number from 3 to 23. In general, such a mAb is neutralizing. In still other embodiments, the mAb for use in the invention binds to a cellular receptor for any one or more of the above-mentioned growth factors.

Native mAbs for use in the invention may be produced from their hybridomas. Genetically engineered mAbs, e.g., chimeric or humanized mAbs, may be expressed by a variety of art-known methods. For example, genes encoding their light and heavy chain V regions may be synthesized from overlapping oligonucleotides and inserted together with available C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the mAbs or other antibodies for use in the invention may be purified according to standard procedures of the art such as microfiltration, ultrafiltration, protein A or G affinity chromatography, size exclusion chromatography, anion exchange chromatography, cation exchange chromatography and/or other forms of affinity chromatography based on organic dyes or the like. Substantially pure antibodies of at least about 90 or 95% homogeneity are preferred, and 98% or 99% or more homogeneity most preferred, for pharmaceutical uses.

### 3. Therapeutic Methods

In a preferred embodiment, the present invention provides a method of treatment with a pharmaceutical formulation comprising a mAb described herein. Pharmaceutical formulations of the antibodies contain the mAb in a physiologically acceptable carrier, optionally with excipients or stabilizers, in the form of lyophilized or aqueous solutions. Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids, carbohydrates, chelating agents, sugars, and other standard ingredients known to those skilled in the art (Remington's Pharmaceutical Science 16th edition, Osol, A. Ed. 1980). The mAb is typically present at a concentration of 1-100 mg/ml, e.g., 10 mg/ml. The mAb can also be encapsulated into carrying agents such as liposomes.

In another preferred embodiment, the invention provides a method of treating a patient with a brain tumor by systemic administration of a mAb, such as a neutralizing anti-HGF mAb or an antibody against a cytokine or its receptor. The patient is preferably human but may be any mammal. By systemic administration, we mean herein a route of administration in which the mAb has general access to the circulatory system, and therefore to the organs of the body, including the blood vessels of the brain. In other words, the mAb is administered on the peripheral side of the blood brain barrier. Examples of systemic administration include intravenous infusion or bolus injection, or intramuscularly or subcutaneously or intraperitoneally. However, systemic administration does not encompass injection directly into the tumor or into an organ such as the brain or its surrounding membranes or cerebrospinal fluid. Intravenous infusion can be given over as little as 15 minutes, but more often for 30 minutes, or over 1, 2, 3 or even 4 or more hours. The dose given is sufficient to cure, at least partially alleviate or inhibit further development of the condition being treated (“therapeutically effective dose”). A therapeutically effective dose preferably causes regression or more preferably elimination of the tumor. A therapeutically effective dosage is usually from 0.1 to 5 mg/kg body weight, for example 1, 2, 3 or 4 mg/kg, but may be as high as 10 mg/kg or even 15 or 20 mg/kg. A fixed unit dose may also be given, for example, 50, 100, 200, 500 or 1000 mg, or the dose may be based on the patient's surface area, e.g., 100 mg/m². A therapeutically effective dosage administered at a frequency sufficient to cure, at least partially alleviate or inhibit further development of the condition being treated is referred to as a therapeutically effective regime. Such a regime preferably causes regression or more preferably elimination of the tumor. Usually between 1 and 8 doses, (e.g., 1, 2, 3, 4, 5, 6, 7 or 8) are administered to treat cancer, but 10, 20 or more doses may be given. The mAb can be administered daily, biweekly, weekly, every other week, monthly or at some other interval, depending, e.g. on the half-life of the mAb, for 1 week, 2 weeks, 4 weeks, 8 weeks, 3-6 months or longer. Repeated courses of treatment are also possible, as is chronic administration.

The methods of this invention, e.g., systemic administration of a mAb such as anti-HGF mAb, especially L2G7 and its variants including humanized L2G7, can be used to treat all brain tumors including meningiomas; gliomas including ependymomas, oligodendrogliomas, and all types of astrcytomas (low grade, anaplastic, and glioblastoma multiforme or simply glioblastoma); medullablastomas, gangliogliomas, schwannomas, chordomas; and brain tumors primarily of children including primitive neuroectodermal tumors. Both primary brain tumors (i.e., arising in the brain) and secondary or metastatic brain tumors can be treated by the methods of the invention. Brain tumors that express Met and/or HGF, especially at elevated levels, are particularly suitable for treatment by systemic administration of a neutralizing anti-HGF antibody such as L2G7 or its variants.

In a preferred embodiment, the mAb is administered together in combination with (i.e., before, during or after) other anti-cancer therapy. For example, the mAb, e.g., an anti-HGF mAb such as L2G7 and its variants, may be administered together with any one or more of the chemotherapeutic drugs known to those of skill in the art of oncology, for example alkylating agents such as carmustine, chlorambucil, cisplatin, carboplatin, oxiplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitoxantrone, vinblastine, vincristine, and Taxol (paclitaxel) or related compounds such as Taxotere®; agents specifically approved for brain tumors including temozolomide and Gliadel® wafer containing carmustine; and other drugs including irinotecan and Gleevec® and all approved and experimental anti-cancer agents listed in WO 2005/017107 A2 (which is herein incorporated by reference). The mAb can be administered in combination with 1, 2, 3 or more of these agents, e.g., in a standard chemotherapeutic regimen. Other agents with which an anti-HGF mAb can be administered include biologics such as monoclonal antibodies, including Herceptin™ against the HER2 antigen, Avastin™ against VEGF, antibodies to the EGF receptor such as Erbitux®, or an anti-FGF mAb, as well as small molecule anti-angiogenic or EGF receptor antagonist drugs such as Iressa® and Tarceva®. In addition, the mAb can be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon).

Although in a preferred embodiment of the invention, the mAb is not linked or conjugated to any other agent, in other embodiments the mAb may be conjugated to a radioisotope, chemotherapeutic drug or prodrug or a toxin. For example, it may be linked to a radioisotope that emits alpha, beta and/or gamma rays, e.g., 90Y, isotopes of iodine such as 131I, or isotopes of bismuth such as 212Bi or 214Bi; to a plant or bacterial protein toxin such as ricin or pseudomonas exotoxin or their fragments such as PE40; to a small-molecule toxin such as compounds related to or derived from calicheamicin, auristatin or maytansine; or to a chemotherapeutic drug such as doxorubin or any of the others chemotherapeutic drugs listed above. Methods of linking such agents to a mAb are well-known to those skilled in the art.

Systemic administration of a mAb, e.g., a neutralizing anti-HGF mAb such as L2G7 or its variants, optionally plus other treatment (e.g., chemotherapy or radiation therapy), can increase the median progression-free survival or overall survival time of patients with certain brain tumors (e.g., glioblastomas) by at least 30% or 40% but preferably 50%, 60% to 70% or even 100% or longer, compared to a control regime without administration of the mAb. If administration of anti-HGF mAb is accompanied by other treatment such as chemotherapy or radiation, the other treatment is also included in the control regime. If anti-HGF mAb is administered without other treatment, the control regime is a placebo or no specific treatment. In addition or alternatively, systemic administration of a mAb, e.g., a neutralizing anti-HGF mAb such as L2G7 or its variants, plus other treatment (e.g., chemotherapy or radiation therapy), may increase the complete response rate (complete regression of the tumor, i.e., remission), partial response rate (a partial response in a patient means partial shrinkage of the tumor size, e.g., by at least 30% or 50%), or objective response rate (complete+partial) of patients with certain brain tumors by at least 30% or 40% of the patients but preferably 50%, 60% to 70% or even 90% or more compared to a control regime without administration of the mAb as described above. Changes in the size of a tumor responsive to treatment can be determined by MRI, CT scanning and the like.

Similarly, when systemically administered to animals (e.g., immunodeficient mice such as nude mice or SCID mice) bearing intracranial xenografts of human glioma tumors, e.g., as described in Example 2 below, the neutralizing anti-HGF mAb or anti-FGF mAb or other mAb will prolong median survival of the animals by at least about 25 or 30 or 40 days, but preferably 50, 60, or 70 days or even longer, and such an extension will be statistically significant. This will be true even when initiation of treatment is delayed until at least 5 or 18 days or longer after tumor cell implantation. Moreover, such treatment will on average shrink the tumors by at least 25% but preferably 50% or even 75%; and the average tumor volume in animals treated with the mAb will be less than 50% or even 25% or 10% of the average tumor volume in control-treated animals. The tumor size will typically be measured 21 or 29 days after tumor cell implantation.

Typically, in a clinical trial (e.g., a phase II, phase II/III or phase III trial), the aforementioned increases in median progression-free survival and/or response rate of the patients treated by administration of a mAb, e.g., an anti-HGF mAb, optionally plus other treatment relative to the patients receiving a control regime without the antibody, are statistically significant, for example at the p=0.05 or 0.01 or even 0.001 level. The complete and partial response rates are determined by objective criteria commonly used in clinical trials for cancer, e.g., as listed or accepted by the National Cancer Institute and/or Food and Drug Administration.

### EXAMPLES

### 1. Generation and In Vitro Properties of Anti-HGF mAbs

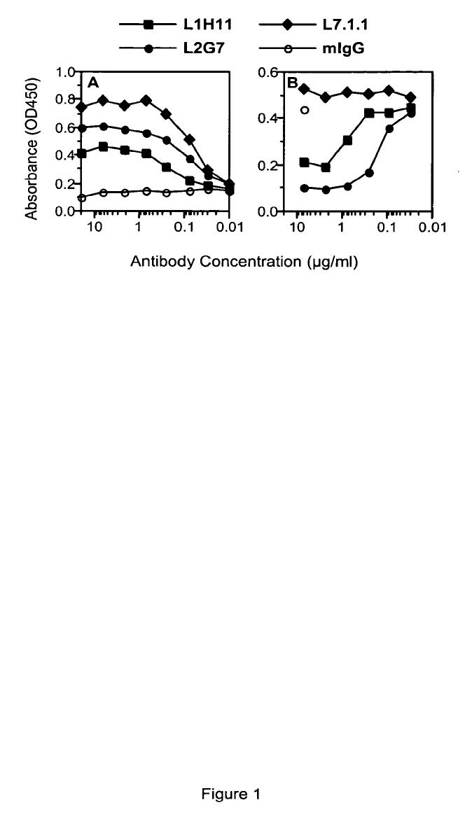

The development of a fully neutralizing anti-HGF mAb L2G7 has been described in U.S. Patent Application Pub. No. US 2005/0019327 A1, which is herein incorporated by reference. In summary, Balb/c mice were extensively immunized with recombinant human HGF by footpad injections, and hybridomas were generated from them by conventional means. Chimeric fusion proteins consisting of HGF fused to Flag peptide (HGF-Flag), and the Met extracellular domain fused to the human IgG1 Fc region (Met-Fc), were produced by conventional recombinant techniques and used to determine the ability of the anti-HGF mAbs to inhibit the binding of HGF to its Met receptor. FIG. 1a demonstrates the ability of three separate anti-HGF mAbs, each recognizing a different epitope to capture HGF in solution. Although the IgG2a mAb L2G7 has intermediate affinity for HGF as judged by binding ability, it is the only mAb identified that completely blocks binding of HGF-Flag to Met-Fc in an ELISA (FIG. 1b). The mAb L2G7 is specific for HGF, as it shows no binding to other growth factors such as VEGF, FGF or EGF.

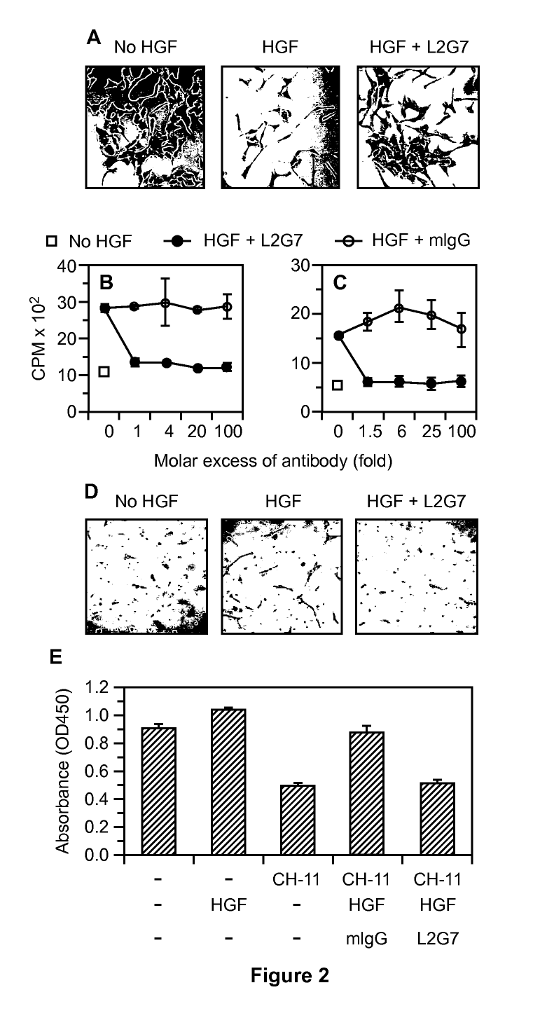

The ability of mAb L2G7 to block HGF binding to Met suggested that it would inhibit all HGF-induced cell responses, but this supposition required verification because the α and β-subunits of HGF mediate different activities (Lokker et al., EMBO J. 11: 2503, 1992; Hartmann et al., Proc. Natl. Acad. Sci. USA 89: 11574, 1992). One important bioactivity of HGF mediated through its α-subunit, from which its alternate name “scatter factor” derives, is the ability to induce cell scattering. FIG. 2a shows that L2G7 is able to completely inhibit HGF-induced scattering of MDCK epithelial cells, a widely used biological assay for quantifying HGF scatter activity. A key biological activity of HGF mediated through its β subunit is mitogenesis of certain cell types. FIG. 2b shows that L2G7 at a 1:1 molar ratio of mAb to HGF completely inhibits HGF-induced 3H-thymidine incorporation in My 1 Lu mink lung epithelial cells. Thus, mAb L2G7 blocks HGF-induced biological activities attributable to both the α- and β-HGF subunits.

Angiogenesis is required for growth of solid tumors. HGF is a potent angiogenic factor (Grant et al., Proc. Natl. Acad. Sci. USA 90: 1937, 1993) and tumor levels of HGF correlate with the vascular density of human malignancies including gliomas (Schmidt. et al. Int. J. Cancer 84: 10, 1999). HGF can also stimulate the production of other angiogenic factors such as VEGF and can potentiate VEGF-induced angiogenesis (Xin et al. Am. J. Pathol. 158, 1111, 2001). Two early steps involved in angiogenesis are endothelial cell proliferation and tubule formation. The effect of L2G7 on HGF-induced proliferation of human umbilical vein endothelial cells (HUVEC) and formation of vessel-like tubules in 3 dimensional collagen gels was therefore determined. Stimulation of HUVEC proliferation by HGF (50 ng/ml, 72 hr) was completely inhibited by L2G7 at a 1.5:1 mAb to HGF molar ratio (FIG. 2c). HUVECs suspended in 3-D collagen gels developed an interconnected branching tubule network after stimulation with HGF (200 ng/ml, 48 hr), while cells treated with HGF plus L2G7 showed little or no such tubule formation (FIG. 2d). Hence L2G7 blocks HGF-induced proliferative and morphogenic aspects of angiogenesis.

HGF protects tumor cells from apoptotic death induced by numerous modalities including DNA-damaging agents commonly used in cancer therapy (Bowers et al. Cancer Res. 60: 4277, 2000; Fan et al. Oncogene 24: 1749, 2005). The majority of human malignant glioma cells express the death receptor FAS, making them susceptible to apoptosis induced by anti-FAS antibody in vitro (Weller et al. J. Clin. Invest. 94: 954, 1994). Thus, the effects of L2G7 on HGF-mediated cytoprotection of U87 glioma cells treated with apoptotic anti-FAS mAb CH-11 were determined. U87 cell viability after CH-11 treatment (24 hr) was reduced to ˜45% of that in untreated controls, an effect that was completely reversed by pre-incubating cells with HGF in the presence of an irrelevant isotype control antibody but not by HGF in the presence of L2G7 (FIG. 2e).

### 2. Effects of Anti-HGF mAb in Glioma Xenograft Tumor Models

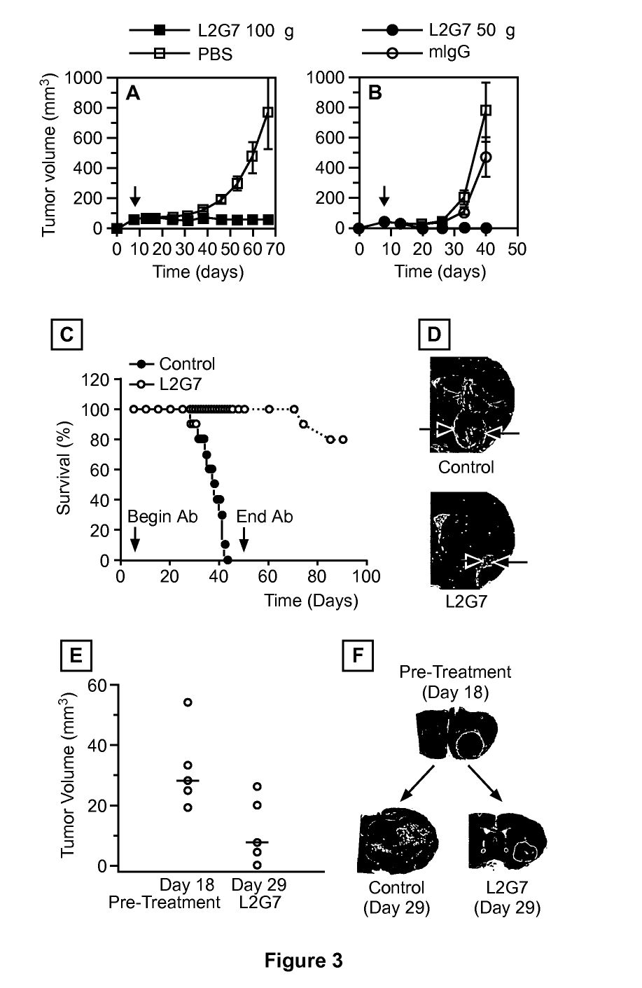

The ability of L2G7 to block multiple tumor-promoting activities of HGF suggested this mAb would have anti-tumor activity against at least HGF+/Met+ human tumors. The majority of gliomas appear to express Met and HGF (Rosen et al. Int. J. Cancer 67: 248, 1996). For the glioma cell lines U87 and U118, Met expression was confirmed by flow cytometric analysis, and ˜20-35 ng/ml HGF in supernatants from 7-day old confluent cultures using an HGF-specific ELISA was detected. The anti-tumor effect of L2G7 in nude mouse models of pre-established U118 and U87 subcutaneous xenografts was determined. L2G7 was administered i.p. twice weekly after tumor sizes had reached ˜50 mm³ as described (Kim et al., Nature 362: 841, 1993, which is herein incorporated by reference). At 100 μg (˜5 mg/kg) per injection, L2G7 completely inhibited growth of U118 tumors (FIG. 3a). In the U87 xenograft model, either 50 μg or 100 μg L2G7 per injection not only inhibited tumor growth but actually caused tumor regression (FIG. 3b). Control mAb (100 μg per injection) only slightly inhibited tumor growth compared to PBS control. L2G7 had no effect on the growth of U251 glioma tumor xenografts, which express Met but do not secrete HGF. These in vivo results demonstrate that L2G7 as a single agent prevents tumor growth by specifically blocking HGF activity.

Next, L2G7 efficacy was examined in mice bearing pre-established intracranial U87 glioma xenografts. Mice were implanted with U87 human malignant glioma cells (100,000 cells/animal) by stereotactic injection to the right caudate/putamen. L2G7 (100 μg/injection, i.p., twice weekly) administered from post-implantation day 5 though day 52 significantly prolonged animal survival (FIG. 3c). In control mice, median survival was 39 days and all mice died from progressive tumors by day 41. In contrast, all mice treated with L2G7 survived through day 70, and 80% survived through day 90, seven weeks after cessation of mAb treatment (FIG. 3c). In sacrificed mice, on day 21 after three doses of L2G7, control tumors were more than 10-fold larger than L2G7-treated tumors (6.6+2.7 mm3 vs. 0.54+0.17 mm3) (FIG. 3d).

To test the mAb efficacy under even more stringent conditions, in a similar experiment initiation of L2G7 treatment was delayed until day 18. A subset of mice (n=5 per group) was sacrificed early in the course of treatment, and tumor volumes were quantified by measuring tumor cross-sectional areas of H&E stained brain sections using computer assisted image analysis. L2G7 induced substantial tumor regression (FIGS. 3e, f). Specifically, pre-treatment tumor volumes on day 18 were 26.7+2.5 mm3 (range 19.5-54 mm3, median 27.9 mm3). On day 29, after 3 doses of L2G7, tumors were only 11.7+5.0 mm3 (range 0-26.2 mm3, median 7.5 mm3), so the tumors had actually regressed or shunk in size on average by 50% or more. Tumor volumes on day 29 from mice treated with isotype-matched control mAb were 134.3+22.0 mm3 (range 71.2-196.8 mm3, median 128 mm³). Hence, tumors treated with control mAb grew nearly 5 fold with a mean volume 12 times larger than the L2G7-treated tumors. In the mice that were not sacrificed (n=10 per group), median survival in the control mice was 32 days and all died by day 42, while none of the L2G7-treated mice died until day 46, and L2G7 extended median survival to day 61. Thus, L2G7 induced tumor regression in mice with very high tumor burdens.

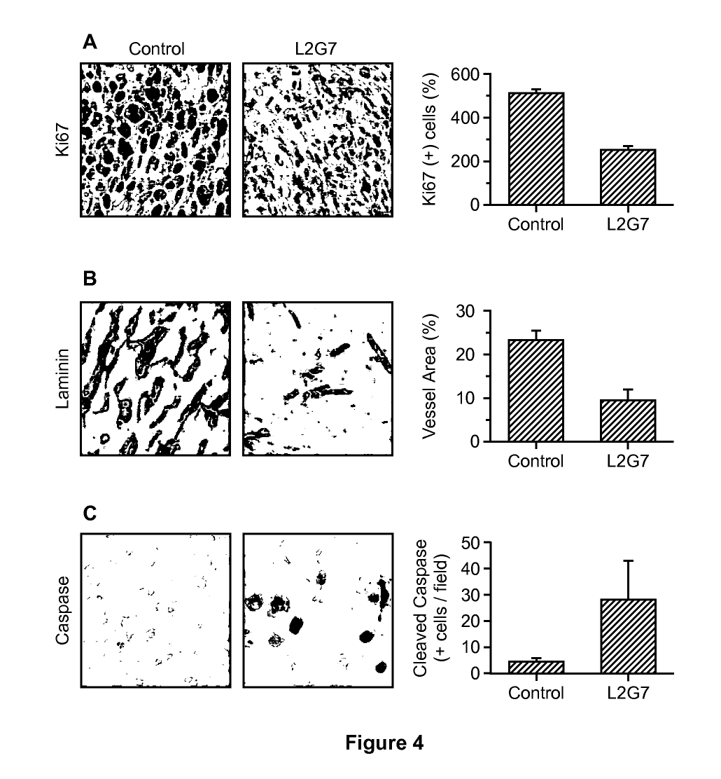

A more detailed analysis of histological sections of intracranial tumors was performed to investigate potential mechanisms of the anti-tumor effects of L2G7 (FIG. 4). Following three doses of L2G7, tumor cell proliferation (Ki-67 index) and angiogenesis (vessel density, i.e. area of anti-laminin stained tumor vessels as percent of tumor area) were reduced by 51% and 62% respectively, while the apoptotic index of tumor cells quantified by the number of activated caspase-3 positive cells was increased 6-fold. The pronounced tumor regression that occurred soon after initiating L2G7 therapy is indicative of a cell death response similar to that observed in human colon tumor Colo 205 xenografts treated with an agonist anti-death receptor 4 (TRAIL1) mAb (Chuntharapai et al. J. Immunol. 166: 4891, 2001).

The results reported here are a striking example of brain tumor responses from a mAb not linked to a toxin or radionuclide. As a comparison, in subcutaneous xenograft models the anti-VEGF murine mAb A4.6.1, which was later humanized to create the drug Avastin®, inhibited growth of the G55 human glioma by only ˜50-60% (Kim et al., Nature 362: 841, 1993), contrasted with essentially complete growth inhibition of the U87 and U118 gliomas by mAb L2G7. In an orthotopic intracranial tumor model, systemic anti-VEGF mAb administered simultaneously with G55 glioma cell implantation prolonged animal survival by only 2-3 weeks (Rubenstein et al. Neoplasia 2: 306, 2000). Similarly, systemic administration of a mAb to a variant of the EGF receptor prolonged median survival of mice with intracranial xenografts of glioma cells transfected with the variant EGF receptor, in general modestly (from 13 to 21 days or from 13 to 19 days, but in one case from 19 days to 58 days; Mishima et al., Cancer Res. 61: 4349, 2001). However, these modest effects were achieved when mAb administration began simultaneously with or shortly after xenograft implantation and hence were likely caused, at least in part, by delaying the initiation of xenograft vascularization, an event that cannot be targeted in patients with pre-existing brain tumors. In contrast, systemic administration of anti-HGF mAb L2G7 prolonged survival and caused tumor regression even when administered on Day 5 or even Day 18 after implantation when the tumors were well-established, and thus corresponds to the situation in human patients.

The pronounced anti-tumor effects of mAb L2G7 are likely due to the unique multifunctional properties of its molecular target HGF, i.e., mitogenic, angiogenic, and cytoprotective (Birchmeier et al. Nat. Rev. Mol. Cell Biol. 4: 915, 2003; Trusolino et al. Nat. Rev. Cancer 4: 289, 2002). The ability of L2G7 to induce glioma regression implicates a cell death response that could result from Fas-mediated apoptosis, which is blocked by HGF binding to Met (Wang et al. Cell. 9: 411, 2002) or from inactivating HGF-induced cytoprotective pathways that involve phosphatidyl inositol 3-kinase, Akt, and NF-kappaB intermediates (Fan et al. Oncogene 24: 1749, 2005). The ability of L2G7 to block the cytoprotective and angiogenic effects of HGF predicts that L2G7 delivered systemically potentiates cytotoxic modalities such as γ-radiation and chemotherapy currently used to treat malignant brain tumors.

Although the invention has been described with reference to the presently preferred embodiments, it should be understood that various modifications can be made without departing from the invention.

All publications, patents and patent applications cited are herein incorporated by reference in their entirety for all purposes to the same extent as if each individual publication, patent and patent application was specifically and individually indicated to be incorporated by reference in its entirety for all purposes.

1. A method of treating a brain tumor in a human patient comprising systemically administering a monoclonal antibody (mAb) to a patient having a brain tumor in the brain of the patient and thereby treating the brain tumor in the brain of the patient.

2. The method of claim 1 wherein the mAb is chimeric, humanized or human.

3. The method of claim 1 wherein the mAb is a neutralizing anti-HGF mAb.

4. (canceled)

5. The method of claim 1 wherein the mAb is administered intravenously.

6. The method of claim 1 wherein the brain tumor is a glioma.

7. The method of claim 6 wherein the brain tumor is a glioblastoma.

8. The method of claim 1 wherein the patient is human.

9. The method of claim 1 wherein the patient is also treated with radiation therapy.

10. The method of claim 1 wherein the mAb is administered together with one or more other active anti-cancer drugs.

11. The method of claim 1 wherein the mAb binds to a growth factor selected from the group consisting of: vascular endothelial cell growth factor (VEGF), nerve growth factor (NGF), brain-derived neurotrophic factor (BDNF), NT-3, transforming growth factor (TGF)-alpha (TGF-α), TGF-β1, TGF-β2, platelet-derived growth factor (PDGF), epidermal growth factor (EGF), heregulin, epiregulin, emphiregulin, neuregulin (NRG)-1alpha (NRG-1α), NRG-1β, NRG-2α, NRG-2β, NRG-3, NRG-4, insulin-like growth factor (IGF)-1 (IGF-1), IGF-2, acidic fibroblast growth factor (FGF) (FGF-1), basic FGF (FGF-2), and FGF-n, where n is any number from 3 to 23.

12. A method of causing regression of a brain tumor in a human patient comprising systemically administering a monoclonal antibody (mAb) to a patient having a brain tumor in the brain of the patient and thereby causing regression of the brain tumor in the brain of the patient.

13. The method of claim 12 wherein the mAb is chimeric, humanized or human.

14. The method of claim 12 wherein the mAb is a neutralizing anti-HGF mAb.

15. (canceled)

16. The method of claim 12 wherein the mAb is administered intravenously.

17. The method of claim 12 wherein the brain tumor is an astrocytoma.

18. The method of claim 17 wherein the brain tumor is a glioblastoma.

19. The method of claim 12 wherein the regression is total regression.

20. The method of claim 12 further comprising treating the patient with radiation therapy.

21. The method of claim 12 wherein the mAb is administered together with one or more other active anti-cancer drugs.

22-23. (canceled)
